(19) United States
(12) Patent Application Publication
(10) Pub. No.: US 2011/0130714 A1
(43) Pub. Date: Jun. 2, 2011

(54) Multifunctional Device And Methods For Tissue Surgery

(76) Inventor: John F. Wells, Tucson, AZ (US)

(21) Appl. No.: 12/629,295

(22) Filed: Dec. 2, 2009

(51) Int. Cl. A61M 1/00

(52) U.S. Cl. 604/28; 604/30

(57) ABSTRACT

A multifunctional device featuring a cartridge having first and second ports; first and second chambers which have outlets disposed at proximal ends connected to tubes that bifurcate to form intake lines and deposit lines, wherein one-way valves are disposed in the intake and deposit lines, wherein pistons are disposed in the chambers which oscillate between moving toward the proximal or distal end, when the piston moves toward the distal end a material can be drawn into the chamber and when the piston moves toward the proximal end the material can be pumped out of the chamber; and wherein the intake lines are formed from bifurcation of a main intake line fluidly connected to the first port; wherein the deposit lines feed into a main deposit line fluidly connected to the second port; wherein the device can pump material from the first port to the second port.

### BACKGROUND OF THE INVENTION

The present invention relates generally to medical devices for tissue surgery (e.g., soft tissue surgery), for example devices that perform functions including but not limited to infusion, aspiration, processing, reinjection, and/or transportation of soft tissue. Examples of soft tissue include but are not limited to body fluids such as adipose tissue, blood, serum, bone marrow, and/or infected tissue and/or unwanted tissue.

Tissue surgery (e.g., soft tissue surgery) may involve a variety of separate processes including infusion of sterile saline and/or medications to a surgical site, aspiration of soft tissue and a portion of the sterile saline and/or medication, processing of the soft tissue for reinjection, reinjection of the soft tissue, and transportation (e.g., transportation of various materials including saline, medication, tissue). Infusion may employ the use of syringes for low volume infusion (e.g., 300 cc or less) or a special infusion pump (e.g., peristaltic or pressure) for high volume infusion (e.g., greater than 300 cc). Equipment for the infusion processes are generally unique, for example they are generally only used for the infusion step of the soft tissue surgery.

Low volume aspiration procedures (e.g., under 300 cc) may employ a syringe (different from the syringe from the infusion step), for example a syringe with an attached needle or cannula. Typically, an incision is made in the skin and the needle/cannula is inserted through the incision site so that the distal end of the needle/cannula lies within the cells to be extracted. The plunger of the syringe is extracted, creating negative pressure within the syringe, there by drawing the cells into the syringe. If this technique is used for larger volumes, a repeat of the steps is required, which exposes the cells to air. Further, there is a heightened risk of infection with the repeated insertion of the needle/cannula. This technique is also detrimental to cell viability. This technique is impractical for high volume aspiration as it is labor intensive and time consuming.

Large volume aspiration procedures (e.g., over 300 cc) may employ a special aspirator that uses air and negative pressure (e.g., a powered vacuum pump attached to a tube) to remove the cells from the surgical site and deposit the cells into a collection chamber. This aspiration technique presents problems, for example the negative pressure may damage the tissue, the equipment may weigh between about 40 to 120 pounds, the equipment may be expensive, the harvested tissue may become exposed to air, which can compromise the viability of the tissue cells, and the high velocity of the tissue aspiration may compromise cell viability. The aspirator equipment, whether for small or large aspiration procedures, is generally unique to the aspiration step.

After soft tissue is obtained, it is processed. For example, the tissue may be cleaned, separated (e.g., via a centrifuge, decanter, multiple syringes), and then transferred to multiple syringes for reinjection. The processing equipment and supplies are generally unique to the processing step. Often the soft tissue must be transferred first from the aspiration equipment and then be transferred to the reinjection equipment.

During reinjection, the soft tissue is often transferred to a series of small syringes. The physician injects (e.g., by hand) the soft tissue to a desired surgical site. Often, the reinjection syringes are taken out and reintroduced to the surgical site a number of times. Controlled reinjection may be difficult for the physician.

As discussed above, the various materials of these processes (e.g., saline, medication, soft tissue) are moved through each step (e.g., infusion, aspiration, processing, reinjection) in a generally disconnected manner because different equipment is used for the different steps.

The present invention features a multifunctional device (e.g., a closed system multifunctional device) for performing steps of soft tissue surgery. The multifunctional device is a single device that utilizes a single set of supplies. Without wishing to limit the present invention to any theory or mechanism, it is believed that the multifunctional device of the present invention is advantageous because the multifunctional device may help eliminate the need to transfer materials (e.g., tissue, saline, medication) from one piece of equipment to another. Mess may be reduced, time may be saved, and contamination may be reduced.

### SUMMARY

The present invention features a multifunctional device comprises (a) a cartridge, (b) a first chamber, wherein a first outlet is disposed at a proximal end of the first chamber, the first outlet is fluidly connected to a first tube, the first tube bifurcates to form a first intake line and a first deposit line, wherein a first one-way valve biased for intake is disposed in the first intake line, and a second one-way valve biased for deposit is disposed in the first deposit line, wherein a first piston is disposed in the chamber, the first piston oscillates between moving toward the proximal end and a distal end of the first chamber, when the first piston moves toward the distal end a material can be drawn into the first chamber through the first outlet and when the first piston moves toward the proximal end the material can be pumped out of the first chamber through the first outlet; and (c) a second chamber, wherein a second outlet is disposed at a proximal end of the second chamber, the second outlet is fluidly connected to a second tube, the second tube bifurcates to form a second intake line and a second deposit line, wherein a third one-way valve biased for intake is disposed in the second intake line and a fourth one-way valve biased for deposit is disposed in the second deposit line, wherein a second piston is disposed in the second chamber, the second piston oscillates between moving toward the proximal end and a distal end of the second chamber, when the second piston moves toward the distal end a material can be drawn into the second chamber through the second outlet and when the second piston moves toward the proximal end the material can be pumped out of the second chamber through the second outlet. In some embodiments, the first intake line and second intake line are formed from bifurcation of a main intake line, the main intake line being fluidly connected to the first port of the cartridge; wherein the first deposit line and the second deposit line feed into a main deposit line, the main deposit line is fluidly connected to the second port of the cartridge; and wherein the device can pump material from the first port to the second port. In some embodiments, the multifunctional device of claim 1, wherein the first piston and second piston move in opposite directions of each other with respect to the proximal and distal end of the respective chambers.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

FIG. 1 is a perspective view of a multifunctional device of the present invention.

FIG. 2A is a first top view and schematic view of an embodiment of a multifunctional device of the present invention.

FIG. 2B is a second top view and schematic view of the multifunctional device of the FIG. 2A, wherein the pistons have moved.

FIG. 3 is an exploded view and schematic view of a portion of the multifunctional device of the present invention, e.g., cartridge.

FIG. 4 is a third top view of the multifunctional device of the FIG. 2A and FIG. 2B.

FIG. 5 is a fourth top view of the multifunctional device of the FIG. 2A and FIG. 2B.

FIG. 6 is a perspective view of an embodiment of a multifunctional device of the present invention.

FIG. 7 is a side view of an alternative embodiment of a multifunctional device of the present invention.

FIG. 8 is a schematic view of an alternative embodiment of the multifunctional device (e.g., cartridge) of the present invention.

### DESCRIPTION OF PREFERRED EMBODIMENTS

The present invention features a multifunctional device 100 (e.g., a closed-system multifunctional device) for performing steps of soft tissue surgery. The steps may include but are not limited to infusion, aspiration, processing, and/or reinjection (and/or transportation).

The multifunctional device 100 comprises a cartridge having a first port (e.g., an “aspiration part”) and a second port (e.g., an “infusion part”). The cartridge is sterile. The cartridge is substantially anaerobic (e.g., minimal air or no air is ever in contact with the materials such as soft tissue, fluid, drugs, etc.). The multifunctional device 100 is a single closed integrated system, which can help provide seamless transportation of materials (e.g., fluids, soft tissue, medication, etc.). Transportation of materials remains within the single cartridge. This results in a clean, continuous, anaerobic, efficient, and rapid movement of materials through the various steps (e.g., infusion, aspiration, processing, reinjection). Without limiting the invention to any mechanism of operations, one of the mechanisms for limiting or preventing air from entering into the system is through the controlled aspiration. For example, when the aspirating cannula of the system is not aspirating a liquid and/or a solid media then aspirating activity stops, as to prevent aspirating air into the system.

Various supplies can be connected (e.g., fluidly connected) to the cartridge. Generally, an IV solution bag, a first cannula (e.g., aspiration cannula), or a collection bag can be attached (e.g., fluidly connected) to the first port of the cartridge, depending on the step of the tissue surgery that is to be performed (see below). Generally, a second cannula (e.g., infusion cannula), the collection bag, and a third cannula (e.g., reinjection cannula) can be attached (e.g., fluidly connected) to the second port of the cartridge, depending on the step of the tissue surgery that is to be performed (see below). The present invention is not limited to the aforementioned supplies. For example, other cannulas, needles, bags, or other supplies may be connected to the cartridge.

### Infusion

For the infusion step, the IV solution bag is fluidly connected to the first port of the cartridge and the second cannula (e.g., infusion cannula) is fluidly connected to the second port of the cartridge. Fluids (e.g., saline, drugs) from the IV solution bag can be directed through the cartridge (e.g., in the first port and out the second port) and through the second cannula (e.g., infusion cannula).

### Aspiration

Prior to the aspiration step and after the infusion step, the first cannula (e.g., aspiration cannula) is fluidly connected to the first port of the cartridge and the collection bag is fluidly connected to the second port of the cartridge. Soft tissue can be aspirated via the first cannula (e.g., aspiration cannula) and delivered through the cartridge (e.g., in the first port and out the second port) to the collection bag.

The multifunctional device 100 can allow for controlled aspiration of the soft tissue. For example in some embodiments, the aspiration step can be stopped before the aspiration cannula is removed from the surgical site. This can help prevent the introduction of unwanted air into the collection bag (e.g., collection chamber).

### Processing

The soft tissue remains in the collection bag, which allows for processing of the soft tissue (e.g., cleaning, decanting, removal of unwanted components). The processing step remains in the collection bag (e.g., a closed system), which can help reduce the risk of contamination (and is fast and efficient). Processing of the soft tissue may include a variety of steps including but not limited to adding solutions (e.g., sterile saline), shaking the bag to mix and scrub materials, allowing the contents to decant, bleeding off undesired components (e.g., blood, serum, tumescent, fluid, saline), removing desired tissue (e.g., adipose tissue), retaining oils and undesired tissues (e.g., cell walls, fibrous materials) in the bag, the like, or a combination thereof.

### Reinjection

The processed soft tissue remains in the collection bag, and the collection bag is connected (e.g., fluidly connected) to the to the first port of the cartridge. The third cannula (e.g., reinjection cannula) is attached to the second port of the cartridge. Soft tissue can be delivered from the collection bag through the cartridge (e.g., in the first port and out the second port) to the third cannula (e.g., reinjection cannula). This allows for continuous reinjection (and is fast and efficient). Risks of contamination may be reduced.

### Flow Rate, Vacuum, and Pressure

Flow rates, and/or vacuums, and/or pressures can be set prior to steps (e.g., infusion, aspiration, reinjection). Flow rates and/or vacuums and/or pressures can be monitored and changed during the steps (e.g., infusion, aspiration, reinjection). Without wishing to limit the present invention to any theory or mechanism, it is believed that the multifunctional device is advantageous because the adjustability of flow rates, vacuums, and pressures can allow for high levels of precision and accuracy. Also, the multifunctional device can operate continuously without being limited by volume. The multifunctional device may operate continuously without the need for interruption.

In some embodiments, the flow rate is between about 0 cc to 100 cc per minute. In some embodiments, the flow rate is between about 100 cc to 500 cc per minute. In some embodiments, the flow rate is between about 500 cc to 1,000 cc per minute. In some embodiments, the flow rate is between about 1,000 cc to 5,000 cc per minute. In some embodiments, the flow rate is between about 5,000 cc to 10,000 cc per minute. In some embodiments, the flow rate more than about 10,000 cc per minute.

In some embodiments, the pressure is between about 0 pounds (e.g., foot pounds) to 5 pounds (e.g., foot pounds). In some embodiments, the pressure is between about 5 pounds (e.g., foot pounds) to 10 pounds (e.g., foot pounds). In some embodiments, the pressure is between about 10 pounds (e.g., foot pounds) to 20 pounds (e.g., foot pounds). In some embodiments, the pressure is between about 20 pounds (e.g., foot pounds) to 30 pounds (e.g., foot pounds). In some embodiments, the pressure is between about 30 pounds (e.g., foot pounds) to 40 pounds (e.g., foot pounds). In some embodiments, the pressure is between about 40 pounds (e.g., foot pounds) to 50 pounds (e.g., foot pounds). In some embodiments, the pressure is more than about 50 pounds (e.g., foot pounds).

In some embodiments, the vacuum is between about 0″ Hg to 5″ Hg (e.g., at sea level). In some embodiments, the vacuum is between about 5″ Hg to 10″ Hg (e.g., at sea level). In some embodiments, the vacuum is between about 10″ Hg to 20″ Hg (e.g., at sea level). In some embodiments, the vacuum is between about 20″ Hg to 29.92″ Hg (e.g., at sea level).

As used herein, the term “about” refers to plus or minus 10% of the reference number. For example, an embodiment wherein the flow rate is about 100 cc per minute includes a flow rate that is between 90 and 110 cc per minute.

The multifunctional device 100 can be constructed to accommodate a variety of surgical requirements (e.g., high or low flow rates, high or low vacuums, etc). The multifunctional device can be constructed in a variety of sizes. For example, in some embodiments, one or more chambers can accommodate a volume between about 0 cc to 2,000 cc. In some embodiments, one or more chambers can accommodate a volume between about 0 cc to 10,000 cc. in some embodiments, one or more chambers can accommodate a volume of more than about 10,000 cc.

### Options

In some embodiments, the multifunctional device 100 further comprises one or more devices for measuring flow rates and/or volumes and/or pressures and/or vacuums. In some embodiments, the multifunctional device 100 further comprises a means of operating the multifunctional device 100 remotely (e.g., remote control systems). In some embodiments, the multifunctional device 100 further comprises one or more foot switches and/or one or more control switches (e.g., on/off switches). In some embodiments, the multifunctional device 100 further comprises a means of measuring altitude (e.g., an altimeter) so as to compensate for vacuum levels/pressure levels.

### Methods

The present invention also features methods, for example methods of performing one or more steps of soft tissue surgery. The steps may include infusion, aspiration, processing, and reinjection. The method comprises obtaining the multifunctional device of the present invention. The method may further comprise fluidly connecting an IV solution bag, a first cannula (e.g., aspiration cannula), or a collection bag to the first port of the cartridge. The method may further comprise fluidly connecting a second cannula (e.g., infusion cannula), the collection bag, or a third cannula (e.g., reinjection cannula) to the second port of the cartridge.

Without wishing to limit the present invention to any theory or mechanism, it is believed that the methods of the present invention allow for performing procedures in a continuous and substantially uninterrupted manner. As used herein, the term “continuous” or “substantially uninterrupted” can be interpreted to mean that no more than about 5 minutes (for example, less than 3 minutes, less than 2 minutes, less than 1 minutes, or less than 30 seconds) elapses between the time the multifunctional device is deactivated (e.g., stopped from pumping) after a first procedure is performed and activated to begin a second procedure. For example, an embodiment wherein the procedure of infusion and the procedure of aspiration are performed in a substantially uninterrupted manner includes an embodiment wherein the multifunctional device is deactivated after the infusion procedure is performed and the multifunctional device is reactivated about 5 minutes later to begin the aspiration procedure.

In some embodiments, the IV solution bag is fluidly connected to the first port of the cartridge and the second cannula (e.g., infusion cannula) is fluidly connected to the second port of the cartridge. Fluids (e.g., saline, drugs) from the IV solution bag can be directed through the cartridge (e.g., in the first port and out the second port) and through the second cannula (e.g., infusion cannula). In some embodiments, the first cannula (e.g., aspiration cannula) is fluidly connected to the first port of the cartridge and the collection bag is fluidly connected to the second port of the cartridge. Soft tissue can be aspirated via the first cannula (e.g., aspiration cannula) and delivered through the cartridge (e.g., in the first port and out the second port) to the collection bag. In some embodiments, the collection bag is connected (e.g., fluidly connected) to the to the first port of the cartridge and the third cannula (e.g., reinjection cannula) is fluidly connected to the second port of the cartridge. Soft tissue can be delivered from the collection bag through the cartridge (e.g., in the first port and out the second port) to the third cannula (e.g., reinjection cannula).

The multifunctional device 100 and methods of the present invention may be used for a variety of applications. Applications may include but are not limited to medical applications in obstetrics or gynecology (OB/GYN), general surgery, brain surgery, ophthalmic surgery, orthopedic surgery, thoracic surgery, gastro intestinal surgery, plastic surgery, and the like. The device 100 and methods of the present invention may be used for procedures in the aforementioned application (or other applications), the procedures including but not limited to: (i) flooding, cleaning, and/or evacuating the abdominal cavity during stomach, colon, or appendectomy surgery or ruptured bowel or intestine surgery; (ii) local anesthesia; (iii) cleaning during gynecologic procedures (e.g., Cesarean section); (iv) thoracic cavity anesthesia; (v) flooding and/or cleaning during lung surgery (e.g., debris, infected materials); (vi) anesthesia, infusing, and/or clean up of orthopedic surgical sites; (vii) infusing, flooding, leaving and/or extracting materials (e.g., blood, serum, and/or undesired materials) within the brain cavity; (viii) infusing, flooding, breaking up, and/or extracting fecal matter from an imported bowel; (ix) infusing, flooding, and/or cleaning of accident-caused wound sites; (x) infusing, flooding, cleaning and/or extracting during cataract surgery.

### Chambers and Flow of Material

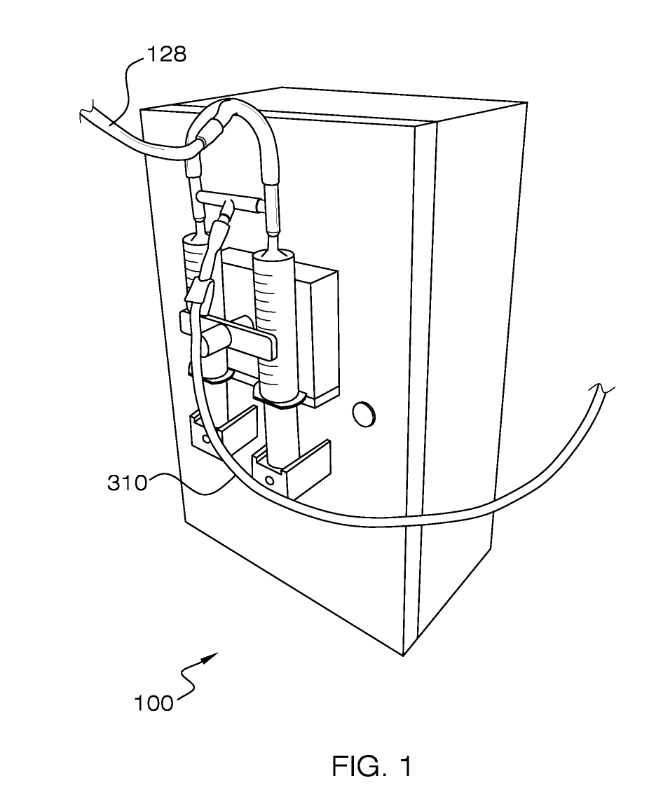

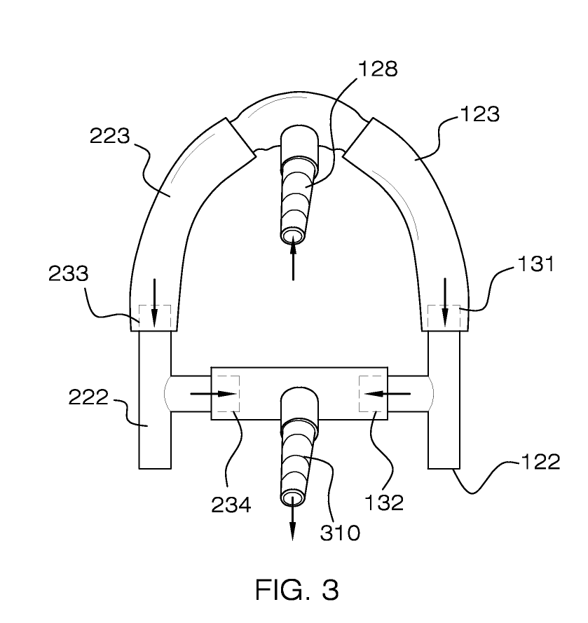

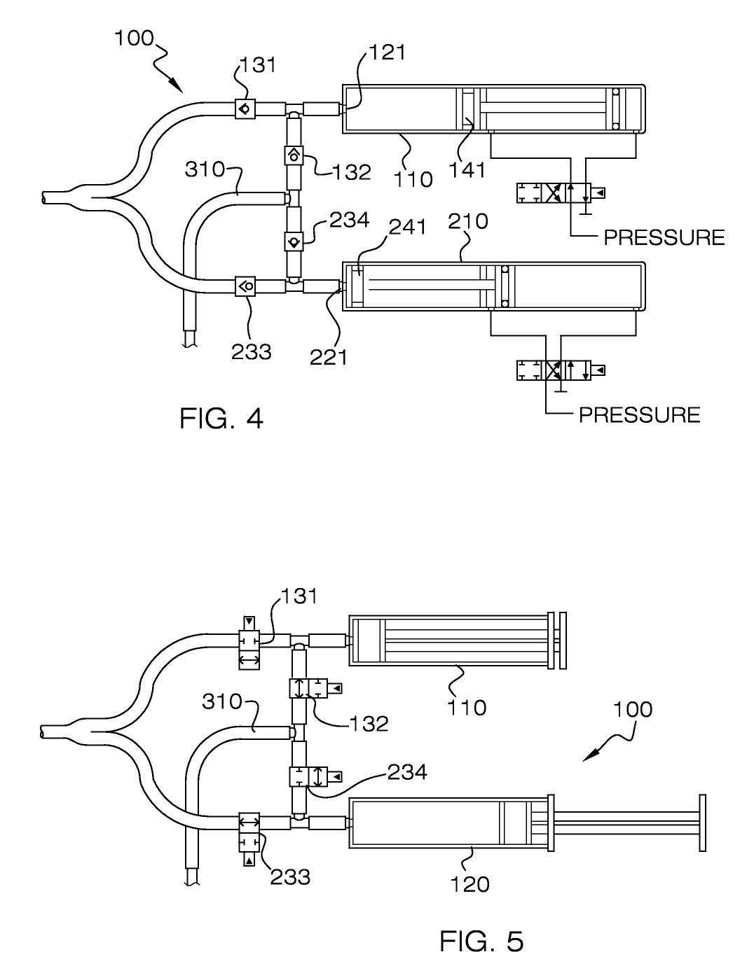

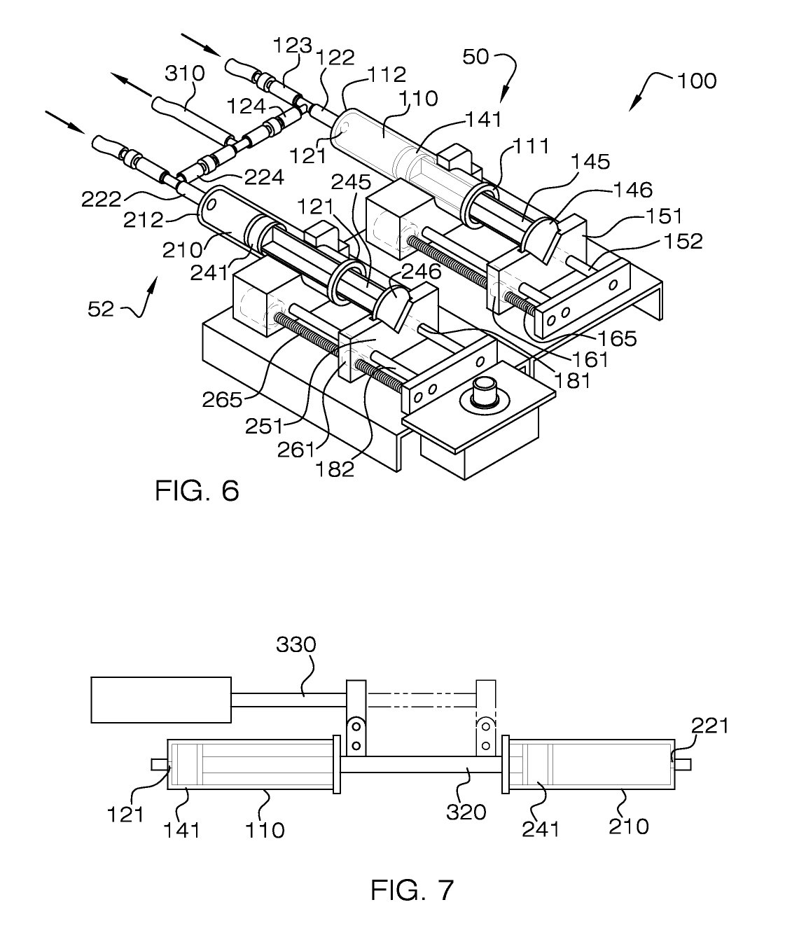

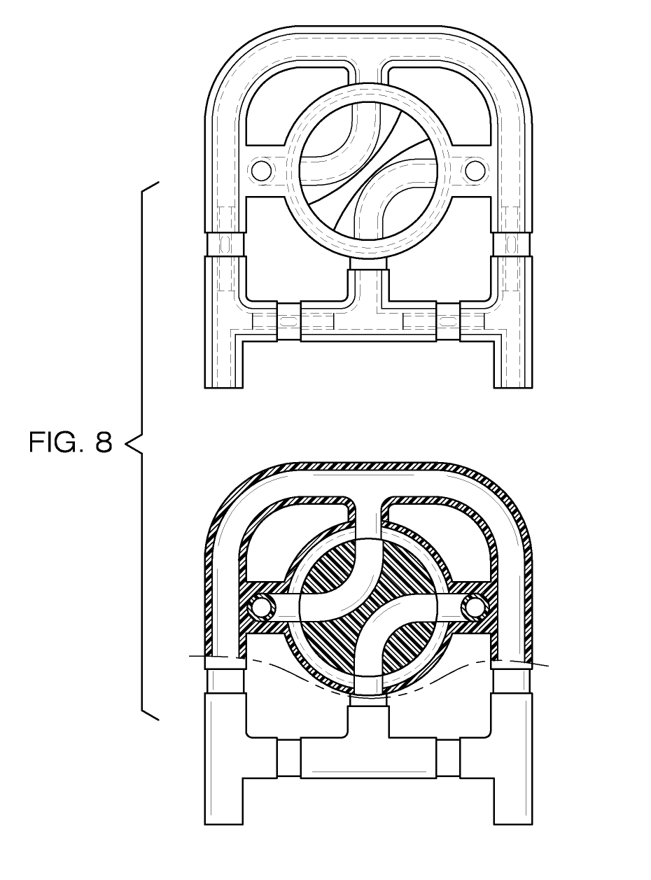

Referring now to FIG. 1-7, the multifunctional device 100 may comprise a first chamber 110 having a distal end 111 and a proximal end 112, wherein a first outlet 121 is located at the proximal end 112. A first piston 141 is disposed in the first chamber 110. The first piston 141 oscillates between moving toward the distal end 111 and the proximal end 112 of the first chamber 110. When the first piston 141 moves toward the distal end 111 of the first chamber 110, a material (e.g., fluid, soft tissue, medication) can be drawn into the first chamber 110 through the first outlet 121. When the first piston 141 moves toward the proximal end 112 of the first chamber 110, the material (e.g., fluid, soft tissue, medication) can be pumped out of the first chamber 110 through the first outlet 121.

The multifunctional device 100 may further comprise a second chamber 210 having a distal end 211 and a proximal end 212, wherein a second outlet 221 is located at the proximal end 212. A second piston 241 is disposed in the second chamber 210. The second piston 241 oscillates between moving toward the distal end 211 and the proximal end 212 of the second chamber 210. When the second piston 241 moves toward the distal end 211 of the second chamber 210, a fluid can be drawn into the second chamber 210 through the second outlet 221. When the second piston 241 moves toward the proximal end 212 of the second chamber 210, the fluid can be pumped out of the second chamber 210 through the second outlet 221.

The first outlet 121 (of the first chamber 110) is fluidly connected to a first tube 122. The first tube 122 bifurcates to form a first intake line 123 and a first deposit line 124. A first one-way valve 131 is disposed within the first intake line 123, wherein the first one-way valve 131 is biased in a first direction (e.g., for intake). A second one-way valve 132 is disposed within the first deposit line 124, wherein the second one-way valve 132 is biased in a second direction (e.g., for deposit).

The second outlet 221 (of the second chamber 210) is fluidly connected to a second tube 222. The second tube 222 bifurcates to form a second intake line 223 and a second deposit line 224. A third one-way valve 233 is disposed within the first intake line 223, wherein the first one-way valve 233 is biased in a first direction (e.g., for intake). A second one-way valve 234 is disposed within the first deposit line 224, wherein the second one-way valve 234 is biased in a second direction (e.g., for deposit).

In some embodiments, the first intake line 123 and second intake line 223 are formed from the bifurcation of a main intake line 128. The main intake line 128 may be fluidly connected to the first port of the cartridge (e.g., where the IV bag and/or aspiration cannula and/or collection bag may be fluidly connected). For example, the IV bag and/or aspiration cannula and/or collection bag may be connected to the first port, and material (e.g. fluid, soft tissue, medication) may be pumped from the IV bag and/or aspiration cannula and/or collection bag through the main intake line 128 and through either the first intake line 123 or the second intake line 223 (and further into the first chamber 110 or into the second chamber 210, respectively).

In some embodiments, the first deposit line 124 and the second deposit line 224 feed into a main deposit line 310. In some embodiments, the main deposit line 310 is fluidly connected to the second port of the cartridge (e.g., where the infusion cannula and/or collection bag and/or reinjection cannula can be fluidly connected). For example, the infusion cannula and/or reinjection cannula and/or collection bag may be connected to the second port, and material (e.g. fluid, soft tissue, medication) may be pumped from either the first chamber 110 or second chamber 120 into the main deposit line 310, and further into the infusion cannula and/or collection bag and/or reinjection cannula.

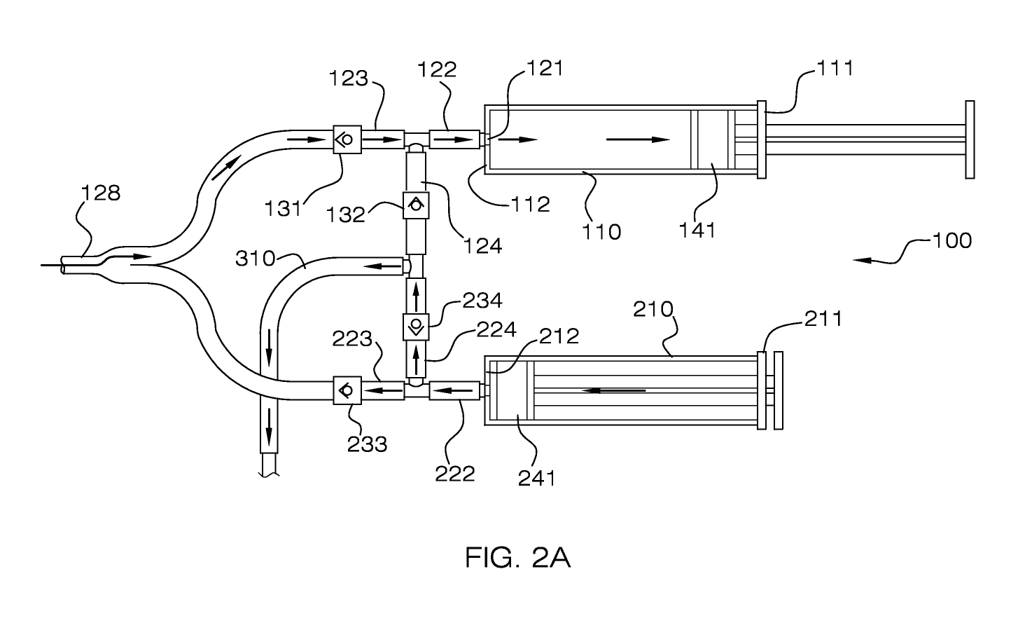

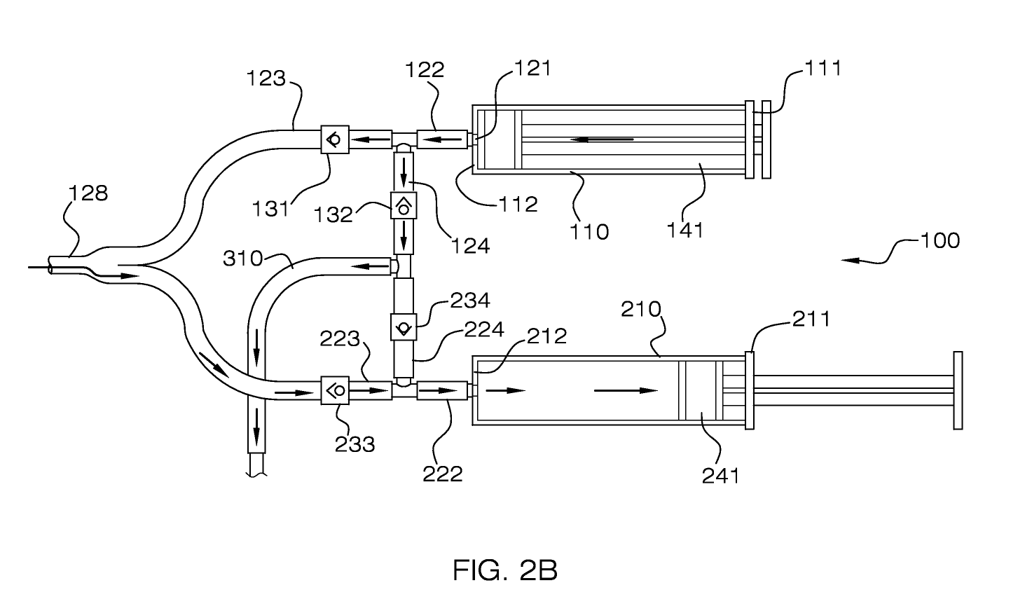

The first piston 141 and the second piston 241 move in opposite directions of each other with respect to the proximal ends and the distal ends of the respective chambers. As shown in FIG. 2A, when the first piston 141 moves toward the distal end 111 of the first chamber 110, a material can be drawn into the first chamber 110 through the first outlet 121. When the second piston 241 moves toward the proximal end 212 of the second chamber 210, the material can be pumped out of the second chamber 210 through the second outlet 221.

As shown in FIG. 2B, when the first piston 141 moves toward the proximal end 112 of the first chamber 110, the material can be pumped out of the first chamber 110 through the first outlet 121. When the second piston 241 moves toward the distal end 211 of the second chamber 210, a material can be drawn into the second chamber 210 through the second outlet 221.

The multifunctional device 100 (e.g., cartridge) may comprise one or more chambers. For example, in some embodiments, the multifunctional device is constructed having a single chamber. The single chamber device may be used for smaller procedures, for example under about 200 cc (e.g., facial operations, etc.). In some embodiments, the multifunctional device is constructed having double chambers (as described above). In some embodiments, the multifunctional device is constructed having three or more chambers.

### Valves

Referring now to FIG. 5, in some embodiments, the first one-way valve 131, second one-way valve 132, third one-way valve 233, and fourth one-way valve 234 are two-way valves such that they can oscillate between an open position and a closed position.

In some embodiments, the valves are solenoid valves that can be electromechanically controlled via a controller chip (e.g., integrated circuit). Solenoid valves and integrated circuits are well known to one of ordinary skill in the art. In some embodiments, the controller chip can read the position of the first piston 141, second piston 241, first valve 131, second valve 132, third valve 233, and fourth valve 234. Based on a program, the controller chip can electrically activate the valves and piston to move them to a particular position (e.g., the open position, the closed position).

For example, in some embodiments, before the first piston 141 moves from the proximal end 112 of the first chamber 110 to the distal end 111 of the first chamber 110 to collect materials in the first chamber 110, the first valve 131 is moved to the open position and the second valve 132 is moved to the closed position. Then, before the first piston 141 moves from the distal end 111 to the proximal end 112 of the first chamber 110 to deposit the materials via the main deposit line 310, the first valve 131 moves to the closed position and the second valve 132 moves to the open position.

In some embodiments, the first one-way valve 131, second one-way valve 132, third one-way valve 233, and fourth one-way valve 234 are reversible such that they can be moved to operate in only a first direction (e.g., biased for intake) or moved to operate in only a second direction (e.g., biased for deposit). For example, the one-way valves can be adjusted to move in a first direction and locked via a locking mechanism in the first direction. Likewise, the one-way valves can be adjusted to move in a second direction and locked in the second direction via a locking mechanism.

In some embodiments, the first one-way valve 131 disposed within the first intake line 123 is biased for intake, the second one-way valve 132 disposed within the first deposit line 124 is biased for deposit, the third one-way valve 233 disposed in the second intake line 223 is biased for intake, and the fourth one-way valve 234 disposed in the second deposit line 224 is biased for deposit. Material (e.g., soft tissue, drugs, fluid) from a first source (e.g., IV bag, collection bag, aspiration cannula) is transported via the first chamber 110 and second chamber 210 into a second source (e.g., infusion cannula, collection bag, reinjection cannula).

In some embodiments, the process may be reversible. The first one-way valve 131 disposed within the first intake line 123 is biased for deposit, the second one-way valve 132 disposed within the first deposit line 124 is biased for intake, the third one-way valve 233 disposed in the second intake line 223 is biased for deposit, and the fourth one-way valve 234 disposed in the second deposit line 224 is biased for intake. Material (e.g., soft tissue, drugs, fluid) from the second source is transported via the first chamber 110 and second chamber 210 into the first source.

### Movement of Pistons

The pistons 141, 241 can be manipulated (e.g., moved, powered) via a variety of mechanisms, and such mechanisms are well known to one of ordinary skill in the art. For example, in some embodiments, the pistons may be powered via a mechanical system, a gas/pressure system, a fluid system, the like, or a combination thereof. The present invention is not limited to the aforementioned examples.

The first piston 141 can be moved toward or away from the first outlet 121 within the first chamber 110 and the second piston 241 can be moved toward or away from the second outlet within the second chamber 210. In some embodiments, the first piston 141 is connected to a first shaft 145, which has a distal end 146 (the distal end 146 not connected to the first piston 141). In some embodiments, the first shaft 145 can be mechanically moved such that it can push or pull the first piston 141 within the first chamber 110. The movement of the first piston 141 is not limited to mechanisms such as a first shaft 145. For example, in some embodiments, the first piston 141 is pushed or pulled using a pressure mechanism.

Likewise, in some embodiments, the second piston 241 is connected to a second shaft 245, which has a distal end 246 (the distal end 246 not connected to the second piston 241). In some embodiments, the second shaft 245 can be mechanically moved such that it can push or pull the second piston 241 within the second chamber 210. The movement of the second piston 241 is not limited to mechanisms such as a second shaft 245. For example, in some embodiments, the second piston 241 is pushed or pulled using a pressure mechanism.

In some embodiments, a connecting shaft 320 links the first piston 141 (of the first chamber 110) and the second piston 241 (of the second chamber 210). The connecting shaft 320 can oscillate between a first direction and a second direction, wherein the first piston 141 and the second piston 241 move in opposite directions of each other with respect to the proximal ends and distal ends of the respective chambers. For example, when the connecting shaft 320 moves in a first direction, the first piston 141 is moved toward the proximal end 112 of the first chamber 110 and the second piston 241 is moved toward the distal end 211 of the second chamber. Likewise, when the connecting shaft 320 is moved in a second direction, the first piston 141 is moved toward the distal end 111 of the first chamber 110 and the second piston 241 is moved toward the proximal end 212 of the second chamber 210.

In some embodiments, the connecting shaft 320 is attached to an auxiliary shaft 330, wherein the auxiliary shaft 330 is for moving the connecting shaft 320. In some embodiments, the auxiliary shaft 330 moves the connecting shaft 320 via an electromechanical means.

### Optional Bases

In some embodiments, the multifunctional device 100 further comprises a first base 151, wherein the first piston 141 is connected to the first base 151 via a first shaft 145. The first base 151 may be mechanically moved along the axis of the first shaft 145 such that it can push or pull the first piston 141 in the first chamber 110 toward or away from the first outlet 121.

In some embodiments, the multifunctional device 100 further comprises a second base 251, wherein the second piston 241 is connected to the second base 251 via a second shaft 245. The second base 251 may be mechanically moved along the axis of the second shaft 245 such that it can push or pull the second piston 241 in the second chamber 210 toward or away from the second outlet 221.

In some embodiments, the first base 151 comprises a first threaded hole 161. A first ball screw 165 can be snugly threaded through the first threaded hole 161 of the first base 151. The first ball screw 165 can rotate in a first direction and a second direction. Rotation of the first ball screw 165 can cause the first base 151 to move along the axis of the first chamber 110 and/or first piston 141 and/or first shaft 145. In some embodiments, the second base 251 comprises a second threaded hole 261. A second ball screw 265 can be snugly threaded through the second threaded hole 261 of the second base 251. The second ball screw 165 can rotate in a first direction and a second direction. Rotation of the second ball screw 265 can cause the second base 251 to move along the axis of the second chamber 210 and/or second piston 241 and/or second shaft 245.

In some embodiments the first ball screw 165 and/or the second ball screw 265 are rotated by an electromechanical means.

In some embodiments, the first base 151 further comprises a guide hole 181, wherein a guide bar 182 can slide through to guide the first base 151 to move along the axis of the first chamber 110 and/or first piston 141 and/or first shaft 145. In some embodiments, the second base 251 further comprises a guide hole 181, wherein a guide bar 182 can slide through to guide the second base 251 to move along the axis of the second chamber 210 and/or second piston 241 and/or second shaft 245.

### Alternative Cartridge

Referring now to FIG. 8, in some embodiments, the multifunctional device further comprises an alternative cartridge. As discussed above, the multifunctional device 100 comprising the aforementioned cartridge may require that components (e.g., cannulas, collection bags, etc.) be switched in between the various procedures (e.g., infusion, aspiration, reinjection, etc.). For example, an operator may need to switch tubing after infusion and before aspiration. The alternative cartridge allows for a completely continuous flow of the procedures (e.g., no need to switch components on or off the ports).

The alternative cartridge comprises a first port and a second port. The alternative cartridge further comprises a knob that rotates in a first direction or a second direction. Rotation of the knob causes the function of the first and second ports to switch. For example, when the knob is in a first position, the first, port is an intake port and the second port is an outlet port. When the knob is in a second position, the second port is an intake port and the first port is an outlet port.

In some embodiments, a vacuum suction cup may be removably attached to the first port and/or the second port (e.g., to accommodate a catheter tip).

The following the disclosures of the following U.S. patents are incorporated in their entirety by reference herein: U.S. Pat. Application No. 2006/0224144; U.S. Pat. Application No. 2008/0091147.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

What is claimed is:

1. A multifunctional device comprising:
(a) a cartridge having a first port and a second port;
(b) a first chamber, wherein a first outlet is disposed at a proximal end of the first chamber, the first outlet is fluidly connected to a first tube, the first tube bifurcates to form a first intake line and a first deposit line, wherein a first one-way valve biased for intake is disposed in the first intake line, and a second one-way valve biased for deposit is disposed in the first deposit line, wherein a first piston is disposed in the chamber, the first piston oscillates between moving toward the proximal end and a distal end of the first chamber, when the first piston moves toward the distal end a material can be drawn into the first chamber through the first outlet and when the first piston moves toward the proximal end the material can be pumped out of the first chamber through the first outlet; and
(c) a second chamber, wherein a second outlet is disposed at a proximal end of the second chamber, the second outlet is fluidly connected to a second tube, the second tube bifurcates to form a second intake line and a second deposit line, wherein a third one-way valve biased for intake is disposed in the second intake line and a fourth one-way valve biased for deposit is disposed in the second deposit line, wherein a second piston is disposed in the second chamber, the second piston oscillates between moving toward the proximal end and a distal end of the second chamber, when the second piston moves toward the distal end a material can be drawn into the second chamber through the second outlet and when the second piston moves toward the proximal end the material can be pumped out of the second chamber through the second outlet,
wherein the first intake line and second intake line are formed from bifurcation of a main intake line, the main intake line being fluidly connected to the first port of the cartridge; wherein the first deposit line and the second deposit line feed into a main deposit line, the main deposit line is fluidly connected to the second port of the cartridge; and wherein the device can pump material from the first port to the second port.

2. The multifunctional device of claim 1, wherein the first piston and second piston move in opposite directions of each other with respect to the proximal and distal end of the respective chambers.

3. The multifunctional device of claim 1, wherein a solution bag, a first cannula, or a collection bag can be fluidly connected to the first port.

4. The multifunctional device of claim 1, wherein a second cannula, a collection bag, a third cannula can be fluidly connected to the second port.

5. The multifunctional device of claim 3, wherein the first cannula is an aspiration cannula.

6. The multifunctional device of claim 4, wherein the second cannula is an infusion cannula.

7. The multifunctional device of claim 4, wherein the third cannula is a reinjection cannula.

8. A method for performing at least two of the following procedures: (a) infusing a surgical site with a material; (b) aspirating a material from the surgical site; (c) processing a material; and (d) reinjecting a material into the surgical site; the method comprises:
(a) obtaining a multifunctional device comprising:
(i) a cartridge having a first port and a second port;
(ii) a first chamber, wherein a first outlet is disposed at a proximal end of the first chamber, the first outlet is fluidly connected to a first tube, the first tube bifurcates to form a first intake line and a first deposit line, wherein a first one-way valve biased for intake is disposed in the first intake line, and a second one-way valve biased for deposit is disposed in the first deposit line, wherein a first piston is disposed in the chamber, the first piston oscillates between moving toward the proximal end and a distal end of the first chamber, when the first piston moves toward the distal end a material can be drawn into the first chamber through the first outlet and when the first piston moves toward the proximal end the material can be pumped out of the first chamber through the first outlet; and
(iii) a second chamber, wherein a second outlet is disposed at a proximal end of the second chamber, the second outlet is fluidly connected to a second tube, the second tube bifurcates to form a second intake line and a second deposit line, wherein a third one-way valve biased for intake is disposed in the second intake line and a fourth one-way valve biased for deposit is disposed in the second deposit line, wherein a second piston is disposed in the second chamber, the second piston oscillates between moving toward the proximal end and a distal end of the second chamber, when the second piston moves toward the distal end a material can be drawn into the second chamber through the second outlet and when the second piston moves toward the proximal end the material can be pumped out of the second chamber through the second outlet;
wherein the first intake line and second intake line are formed from bifurcation of a main intake line, the main intake line being fluidly connected to the first port of the cartridge; wherein the first deposit line and the second deposit line feed into a main deposit line, the main deposit line is fluidly connected to the second port of the cartridge; and wherein the device can pump material from the first port to the second port; and
(b) performing at least two of the following steps:
(i) fluidly connecting a solution bag with a material to the first port of the cartridge and fluidly connecting a second cannula to the second port of the cartridge; then activating the multifunctional device so as to infuse the surgical site with the material from the solution bag, the material being transported from the solution bag through the first port and through the second port and through the second cannula to the surgical site;
(ii) fluidly connecting a first cannula to the first port of the cartridge and fluidly connecting a collection bag to the second port of the cartridge; then activating the multifunctional device so as to aspirate a material from the surgical site, the material being transported from the surgical site through the first cannula and through the first port and through the second port to the collection bag;
(iii) subjecting a material in the collection bag to a processing treatment; and
(iii) fluidly connecting a collection bag to the first port of the cartridge and fluidly connecting a third cannula to the second port of the cartridge; then activating the multifunctional device so as to reinject a material into the surgical site, the material being transported from the collection bag through the first port and through the second port and through the third cannula to the surgical site.

9. The method of claim 8, wherein the procedures are performed in a continuous or substantially uninterrupted manner.

10. The method of claim 8, wherein the material includes tissue, fluid, drugs, or a combination thereof.

11. The method of claim 8, wherein the method is substantially anaerobic.

12. The method of claim 8, wherein the processing treatment includes adding a solution to the material in the collection bag; shaking the material in the collection bag; decanting the material in the collection bag; bleeding off an undesired component from the material in the collection bag; removing a desired tissue from the material in the collection bag; retaining a desired component in the material in the collection bag; or a combination thereof.

13. The method of claim 8, wherein the multifunctional device has a flow rate, the flow rate can be set before a step is performed and the flow rate can be monitored or changed during the step.

14. The method of claim 8, wherein the multifunctional device has a flow rate, the flow rate is between about 0 cc to 10,000 cc per minute.

15. The method of claim 8, wherein the multifunctional device has a pressure, the pressure can be set before a step is performed and the flow rate can be monitored or changed during the step.

16. The method of claim 8, wherein the multifunctional device has a pressure, the pressure is between about 0 foot pounds to 50 foot pounds.

17. The method of claim 8, wherein the multifunctional device has a vacuum, the vacuum can be set before a step is performed and the flow rate can be monitored or changed during the step.

18. The method of claim 8, wherein the multifunctional device has a vacuum, the vacuum is between about 0″ Hg to 29.92″ Hg as measured at sea level.
